SEQUENCE LISTING (8 sequences)

SEQ ID NO: 1 - DNA, 20 bp, Artificial sequence
  PCR primer
gctgcttccaaacctttgac

SEQ ID NO: 2 - DNA, 19 bp, Artificial sequence
  PCR primer
ttctccacagccacatgag

SEQ ID NO: 3 - DNA, 20 bp, Artificial sequence
  PCR primer
ggaccaagaccatccaattc

SEQ ID NO: 4 - DNA, 20 bp, Artificial sequence
  PCR primer
ggcataacgcactaggtttg

SEQ ID NO: 5 - DNA, 20 bp, Artificial sequence
  PCR primer
gctttccgaattcactggag

SEQ ID NO: 6 - DNA, 20 bp, Artificial sequence
  PCR primer
ttgcacctcagggaagaatc

SEQ ID NO: 7 - DNA, 20 bp, Artificial sequence
  PCR primer
ggcaaagtggagattgttgc

SEQ ID NO: 8 - DNA, 20 bp, Artificial sequence
  PCR primer
aatttgccgtgagtggagtc

(19) United States
(12) Patent Application Publication
(10) Pub. No.: US 2018/0147236 A1
(43) Pub. Date: May 31, 2018

(54) METHOD OF SUPPRESSING INFLAMMATION AND PROMOTING REPAIR OF INJURED NEURAL TISSUES WITH CARBOXYL-FUNCTIONALIZED POLYURETHANE NANOPARTICLES

(71) Applicant: NATIONAL TAIWAN UNIVERSITY, Taipei City (TW)

(72) Inventors: Shan-Hui Hsu, Taipei City (TW); Yen-Jang Huang, Taipei City (TW); Po-Hao Huang, Taipei City (TW); Yi-Hua Hsu, Taipei City (TW)

(21) Appl. No.: 15/361,300

(22) Filed: Nov. 25, 2016

(51) Int. Cl. A61K 31/785; A61K 9/00

(52) U.S. Cl. CPC A61K 31/785; A61K 9/0014

(57) ABSTRACT

The present invention provides a method of suppressing inflammation and promoting repair of an injured neural tissue in a subject in need thereof, including administering to the injured neural tissue an effective amount of a carboxyl-functionalized polyurethane nanoparticle. The method of the present invention significantly suppresses immune responses by inhibiting pro-inflammatory cytokine production and facilitates neurological recovery, and thus it may be applied directly in patients going through neurosurgery.

### BACKGROUND OF THE INVENTION

### 1. Field of the Invention

The present invention relates to a method for immune intervention and tissue repair. Particularly, the present invention relates to a method of suppressing inflammation and promoting repair of injured neural tissues with carboxyl-functionalized polyurethane nanoparticles.

### 2. The Prior Art

Brain surgery has been widely performed in patients with neurological diseases, such as brain tumors, neural vascular diseases, and head trauma. In traditional brain surgery, either the trans-cortical or trans-sulcal route is performed with brain retraction, during which neurosurgeons use metallic retractors to separate normal brain tissues. This operation inevitably leads to brain injuries and intra-cerebral hemorrhage (ICH, i.e. bleeding from brain vessels into the brain parenchyma). Other causes, including traumatic brain injury or hemorrhagic stroke, also cause ICH.

The leaked blood is one of the most toxic substances which the brain can be exposed to. The degraded blood cells release their breakdown products, such as thrombin and ferrous iron, which subsequently activate microglia and result in neuroinflammation. The activated microglia initiates a pathogenic cascade involving release of pro-inflammatory cytokines. Among these cytokines, interleukin-1 beta (IL-1β) and tumor necrosis factor alpha (TNF-α) have been revealed to cause DNA fragmentation followed by neuronal death. Therefore, due to induction of neuroinflammation following ICH, brain surgery itself may worsen brain injuries.

The treatment of ICH usually comes with unacceptably high death rate and complications. The fundamental problem is that surgeons need to go through normal brain tissues in order to remove the deep seated blood clot. The difficulty of avoiding damage to normal brain tissues during this treatment increases mortality of traditional brain surgery to 36% and causes nearly 74% unfavorable outcome in patients.

Accordingly, there is an urgent need to develop a strategy to reduce brain injuries of patients going through brain surgery and the associated high mortality.

### SUMMARY OF THE INVENTION

As a result, the present invention provides a method of suppressing inflammation and promoting repair of an injured neural tissue in a subject in need thereof, comprising administering to the injured neural tissue an effective amount of a carboxyl-functionalized polyurethane nanoparticle.

In one embodiment of the present invention, the injured neural tissue is an injured cerebral cortex.

In another embodiment of the present invention, the carboxyl-functionalized polyurethane nanoparticle comprises a main chain of polyurethane comprising a hard segment and a soft segment, wherein the hard segment is formed by reaction of a diisocyanate and a chain extender, and the soft segment is a biodegradable oligodiol. Preferably, the biodegradable oligodiol is polycaprolactone diol; the diisocyanate is isophorone diisocyanate; and the chain extender is selected from the group consisting of 2,2-bis(hydroxymethyl)propionic acid and ethylenediamine.

In yet another embodiment of the present invention, the carboxyl-functionalized polyurethane nanoparticle has a zeta potential of about −70 to −50 mV, and it is at a size of about 34-64 nm.

In still another embodiment, the carboxyl-functionalized polyurethane nanoparticle is administered topically at a dose of at least 10 mg/kg.

In yet another embodiment, the carboxyl-functionalized polyurethane nanoparticle reduces activated nuclear factor kappa B (NF-κB) and suppresses gene expression of pro-inflammatory cytokines, including interleukin-1 beta (IL-1β), interleukin-6 (IL-6), and tumor necrosis factor alpha (TNF-α).

The method of the present invention utilizes the carboxyl-functionalized polyurethane nanoparticle as a local immune intervention agent, which can be applied, during surgery, directly to the site of brain retraction and injured neural tissues. The carboxyl-functionalized polyurethane nanoparticle is demonstrated to both effectively inhibit inflammatory responses and promotes repair of the injured neural tissues. Therefore, the method of the present invention may not only reduce neuroinflammation, resulting from the original neurological diseases or the neurosurgery, and the mortality of neurosurgery patients but also enhance patients' neurological recovery. Moreover, since the carboxyl-functionalized polyurethane nanoparticle may be used for immunosuppression without carrying any other anti-inflammatory agents, the method of the present invention may lower the risk of infection due to systemic application of conventional anti-inflammatory drugs such as steroids and the resulting pan-inhibition of the immune response.

The present invention is further explained in the following drawings and examples. It is understood that the examples given below do not limit the scope of the invention, and it will be evident to those skilled in the art that modifications can be made without departing from the scope of the appended claims.

### BRIEF DESCRIPTION OF THE DRAWINGS

The present invention will be apparent to those skilled in the art from the following detailed description of the preferred embodiments, with reference to the attached drawings, in which:

FIG. 1 shows the synthetic process of the carboxyl-functionalized polyurethane nanoparticle (PU NP) of the present invention, including three types of PU NPs, namely PU1 NP, PU2 NP, and PU3 NP, with increasing surface carboxyl groups;

FIG. 2A shows the mRNA expression levels of IL-1β in macrophages incubated with either one of the three types of PU NPs (PU1 NP, PU2 NP, and PU3 NP) and then treated with lipopolysaccharide (LPS);

FIG. 2B shows the mRNA expression levels of IL-6 in macrophages incubated with either one of the three types of PU NPs (PU1 NP, PU2 NP, and PU3 NP) and then treated with LPS, *p<0.05;

FIG. 2C shows the mRNA expression levels of TNF-α in macrophages incubated with either one of the three types of PU NPs (PU1 NP, PU2 NP, and PU3 NP) and then treated with LPS, *p<0.05;

FIG. 3A shows detection of phosphor-nuclear factor kappa B (pNF-κB) in macrophages incubated with PU3 NP and then treated with LPS by western blot analysis, *p<0.05;

FIG. 3B shows co-localization of pNF-κB and LC3 in the fluorescence microscopy images of macrophages incubated with PU3 NP and then treated with LPS; the scale bar represents 20 μm;

FIG. 4A shows a cross-sectional view of rat bregma with a rectangular opening;

FIG. 4B depicts a subdural hemorrhage (SDH)-induced injured area as a dotted region in a coronal section of a rat brain;

FIG. 4C shows western blot results of TNF-α in an injured or normal side of a cortex section of a rat after SDH surgery and PU3 NP treatment;

FIG. 4D shows western blot results of IL-1β in an injured or normal side of a cortex section of the rat after SDH surgery and PU3 NP treatment;

FIG. 5A shows a micrograph and enlarged views of parts of the micrograph of a cortex section of a rat after SDH surgery;

FIG. 5B shows a micrograph and enlarged views of parts of the micrograph of a cortex section of a rat after SDH surgery and treatment with PU3 NP;

FIG. 6A shows a brain slice of a middle cerebral artery occlusion (MCAO) rat model without PU NP treatment;

FIG. 6B shows a brain slice of a MCAO rat model with PU3 NP treatment at a dose of 20 mg/kg after 1.5 hours of reperfusion; and

FIG. 6C shows a brain slice of a MCAO rat model with PU3 NP treatment at a dose of 10 mg/kg after 3 hours of reperfusion.

### DETAILED DESCRIPTION OF THE PREFERRED EMBODIMENT

### Definition

Numerical quantities given herein are approximate, and experimental values may vary within 20 percent, preferably within 10 percent, and most preferably within 5 percent. Thus, the terms “about” and “approximately” refer to within 20 percent, preferably within 10 percent, and most preferably within 5 percent of a given value or range.

The present invention relates to a method of suppressing inflammation and promoting repair of an injured neural tissue in a subject in need thereof, including the step of administering to the injured neural tissue an effective amount of a carboxyl-functionalized polyurethane nanoparticle. The anti-inflammatory effect of the carboxyl-functionalized polyurethane nanoparticle on macrophage-mediated immune responses and its neuroprotective effect in animal models are further described in the following examples.

### Methods and Materials

### Preparation of the Carboxyl-Functionalized Polyurethane Nanoparticles

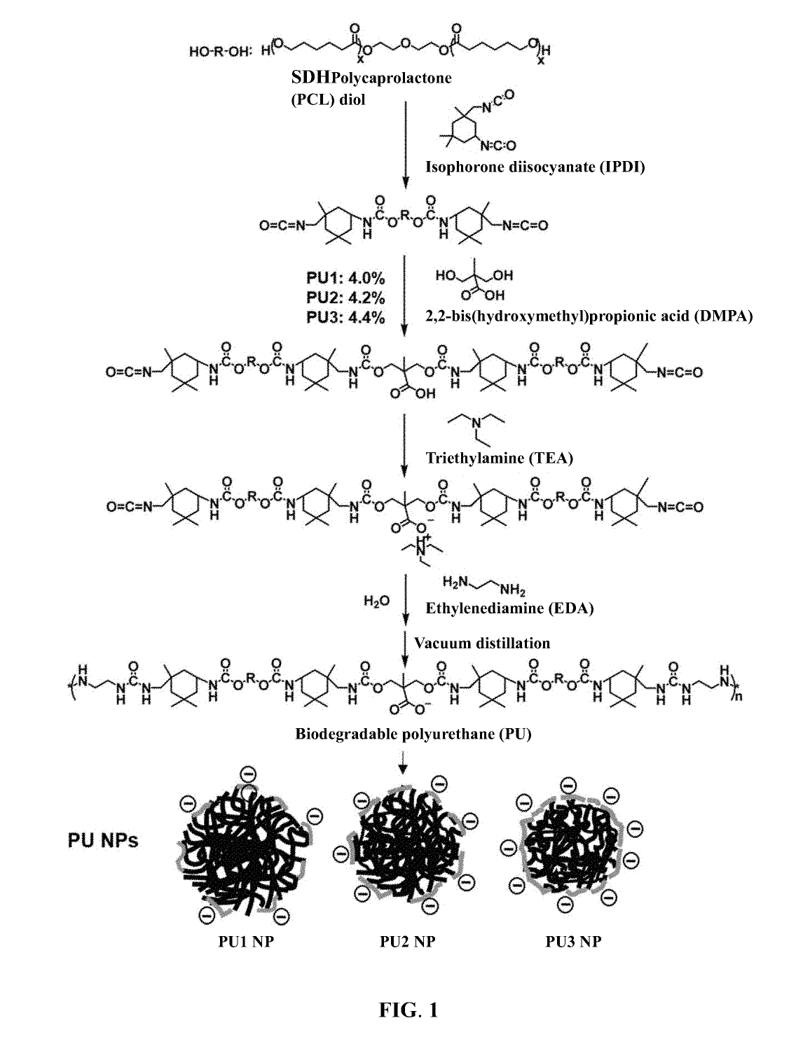

The carboxyl-functionalized polyurethane nanoparticle (hereinafter abbreviated as PU NP) comprises a main chain of polyurethane, which contains a hard segment and a soft segment. The PU NPs used in the following examples were synthesized as follows and as shown in FIG. 1. Polycaprolactone diol (PCL diol, Mw about 2 kDa, Sigma), used to form the soft segment, was added to a flask and mechanically homogenized at 180 rpm at 75° C. to a homogenized liquid. Next, isophorone diisocyanate (IPDI, Evonik Degussa GmbH), used to form one part of the hard segment, was added to react with the homogenized liquid under nitrogen at 75° C. for 3 hours. The ionic chain extender 2,2-bis(hydroxymethyl)propionic acid (DMPA, Sigma) and methyl ethyl ketone (MEK, J.T. Baker) were then added to react at 75° C. for 1 hour. When the temperature was decreased to 45° C., triethylamine (TEA, RDH) was added to neutralize the carboxylic group of DMPA for 30 minutes. The neutralized prepolymer was then dispersed in deionized water by vigorous stirring. Subsequently, a second chain extender ethylenediamine (EDA), used to form another part of the hard segment, was added to react for another 30 minutes. The residual MEK and TEA were removed by vacuum distillation. The final product was PU NP suspended in distilled water with the solid content of the dispersion being about 30 wt %. Three types of PU NPs, termed PU1 NP, PU2 NP, and PU3 NP, were synthesized with increasing ionic contents where DMPA ratios were 4.0, 4.2, and 4.4 wt % with respect to the PU molecule. For each type of PU NP, the stoichiometric ratios of IPDI:PCL diol:DMPA:EDA were 3.47:1:0.95:1.52 for PU1 NP, 3.52:1:1:1.52 for PU2 NP, and 3.57:1:1.05:1.52 for PU3 NP. In FIG. 1, the black segments and the gray segments of the three types of PU NPs represent the soft segments and the hard segments, respectively.

### Cell Culture

Mouse macrophage cell line J774A.1 were cultured in Dulbecco's modified Eagles medium (DMEM, Gibco) supplemented with 10% fetal bovine serum (FBS, Gibco) and 1% penicillin-streptomycin-amphotericin (PSA) solution (Gibco). Cells were incubated at 37° C. in a humidified atmosphere containing 5% CO₂ and subcultured twice a week. Upon experimentation, cells were seeded in 6-well culture plates.

Quantitative Real Time Reverse Transcription-Polymerase Chain Reaction (qRT-PCR) Analysis

The mRNA expression levels of pro-inflammatory cytokines were determined by qRT-PCR. Trizol reagent (Invitrogen) was used to extract total RNA from cells according to the manufacture's instruction. Total RNA (1 μg) was reverse-transcribed into cDNA by RevertAid First Strand cDNA Synthesis Kit (MBI Ferments, St Leon-Rot, Germany) qRT-PCR was conducted in a Chromo 4 PTC200 Thermo Cycler (MJ Research, USA) using the DyNAmo Flash SYBR Green qPCR kit (Finnzymes Oy, Espoo, Finland) and primers for IL-1β, IL-6, TNF-α, and glyceraldehyde 3-phosphate dehydrogenase (GAPDH). The gene expression levels were normalized to those of GAPDH. The normalized value was shown as the relative ratio to that in the mock group. The primer sequences are listed in TABLE 1.

TABLE 1

| | | Primer |
|---|---|---|
| | | annealing |
| | | temperature |
| Gene | Forward primer and Reverse primer | (° C.) |
| IL-1β | F: GCTGCTTCCAAACCTTTGAC (SEQ ID NO: 1) | 62 |
| | R: TTCTCCACAGCCACATGAG (SEQ ID NO: 2) | |
| IL-6 | F: GGACCAAGACCATCCAATTC (SEQ ID NO: 3) | 62 |
| | R: GGCATAACGCACTAGGTTTG (SEQ ID NO: 4) | |
| TNF-α | F: GCTTTCCGAATTCACTGGAG (SEQ ID NO: 5) | 62 |
| | R: TTGCACCTCAGGGAAGAATC (SEQ ID NO: 6) | |
| GAPDH | F: GGCAAAGTGGAGATTGTTGC (SEQ ID NO: 7) | 60 |
| | R: AATTTGCCGTGAGTGGAGTC (SEQ ID NO: 8) | |

### Western Blot Analysis

J774.A1 cells were seeded at a density of 2.5×10⁵ cells per well in a 6-well culture plate and cultured for 24 hours. Cells were incubated with PU NP for 30 minutes and then were treated with lipopolysaccharides (LPS). After the 24-hour treatment, cells were harvested and lysed in a lysis buffer containing 20 mM Hepes (pH 7.5), 420 mM NaCl, 1.5 mM MgCl₂, 0.1% NP-40, and protease inhibitor cocktail (Sigma). Sample proteins were separated by 15% sodium dodecyl sulfate polyacrylamide gel electrophoresis (SDS-PAGE) and then transferred to a nitrocellulose membrane. Proteins of interest were detected with the rabbit polyclonal anti-LC3 antibody (Genetex), rabbit monoclonal anti-phospho-NF-κB (anti-pNF-κB) antibody (Genetex), rabbit monoclonal anti-GAPDH antibody (Genetex), anti-TNF-α antibody (Abcam, UK), anti-IL-1β antibody (Abcam, UK), and anti-beta actin (anti-β-actin) antibody (Abcam, UK). The intensities of protein bands were detected by using Labwork software (UVP). The related protein expression was quantified by using the software ImageJ.

### Immunocytochemistry

After LPS and PU NP treatment, J774.A1 cells were washed with phosphate buffered saline (PBS, pH 7.4, sodium chloride 137 mM, potassium chloride 2.7 mM, sodium hydrogen phosphate 10 mM, and potassium dihydrogen phosphate 1.8 mM) and fixed in 4% paraformaldehyde at room temperature for 10 mM Cells were washed with 1% Tween/PBS and incubated with rabbit polyclonal anti-LC3 antibody (Genetex) for autophagosomes and rabbit monoclonal anti-pNF-κB antibody (Genetex) for NF-κB activation. The second antibodies were goat anti-rabbit IgG labeled with FITC (Santa Cruz) for LC3 and donkey anti-rabbit IgG labeled with Alexa Fluor 594 (Biolegend) for pNF-κB. Cells were analyzed using a fluorescence microscope.

### Animal Studies

For experiments with rat models, 12 adult male Sprague-Dawley rats (8-10 weeks old, weighing 280-350 g; purchased from National Laboratory Animal Center, Taiwan) were used. The rats were housed in a specific-pathogen-free environment with a 12:12-hour light:dark cycle and controlled humidity (55%) and temperature (22° C.). The rats were allowed to freely access food and water.

Histopathological Examination with Hematoxylin and Eosin (H&E) Staining

The rats were transcardially perfused with PBS-buffered 4% paraformaldehyde (pH 7.4). Their brains were then removed and post-fixed for 24 hours before embedded in paraffin. The paraffinized brains were sectioned at 5 um, deparaffnized, and then stained with hematoxylin and eosin. Briefly, the sections were immersed in a hematoxylin solution for 10 minutes, differentiated in PBS for 3 minutes, and immersed in an eosin solution for 1 minute. Images were photographed by a microscope (Olympus BX51, Olympus, Tokyo, Japan).

### 2,3,5-Tetraphenyltetrazolium Chloride (TTC) Staining

The brains of the sacrificed rats were quickly isolated, placed in a brain matrix, and sectioned into serial 2-mm-thick coronal slices with blades. The brain slices were then immersed in 2% TTC solution at 37° C. for 30 minutes for examination.

### Statistical Analysis

Data from multiple samples are expressed as the mean±standard deviation. Three similar experiments at least were performed independently for each experiment.

### Example 1

### Characterization of the Carboxyl-Functionalized Polyurethane Nanoparticles

The diameter and zeta potential of the PU NPs of the present invention were determined by light scattering. The PU1 NP, which contained 4.0% DMPA, had a hydrodynamic diameter of 64.3±0.8 nm and a zeta potential of −48.1±3.9 mV. The PU2 NP, which contained 4.2% DMPA, had a smaller hydrodynamic diameter of 41.9±0.7 nm and a more negative zeta potential of −63.1±3.5 mV. The PU3 NP, which contained 4.4% DMPA, had the smallest hydrodynamic diameter of 34.0±1.3 nm and the most negative zeta potential of −70.8±0.8 mV. The result indicates that PU3 NP, which has the highest DMPA content among the three types of PU NPs, has the highest surface carboxyl-modification.

### Example 2

### Anti-Inflammatory Effect of the Carboxyl-Functionalized Polyurethane Nanoparticles Via Inhibiting Pro-Inflammatory Cytokine Expression

The anti-inflammatory effect of the PU NP of the present invention was verified by determining gene expression levels of pro-inflammatory cytokines after J774A.1 macrophages were first incubated with PU1 NP, PU2 NP, or PU3 NP, for 30 minutes and then treated for 24 hours with 100 ng/ml LPS, an endotoxin eliciting strong immune responses in cultured cells and animals.

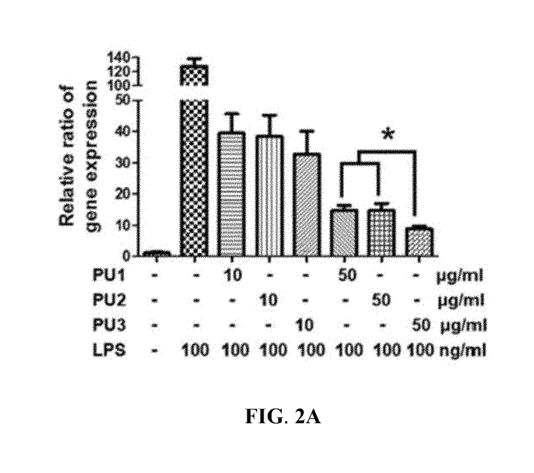

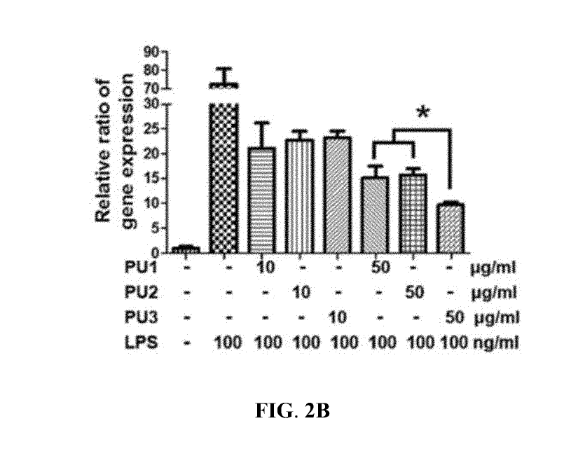

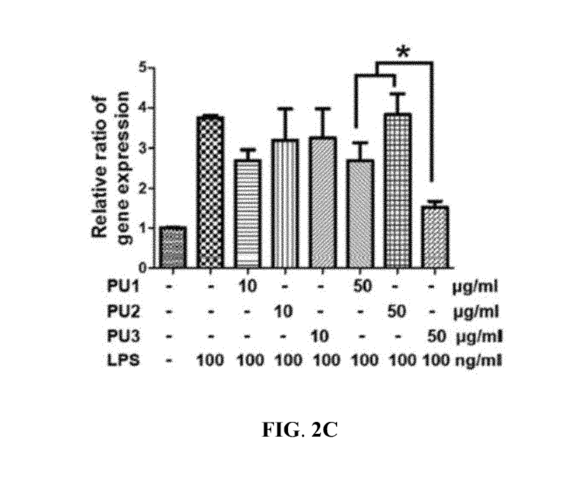

As shown in FIGS. 2A-2C, the mRNA expression levels of IL-1β, IL-6, and TNF-α in the macrophages treated only with LPS were increased when compared with the cells with no treatment (the mock group). However, the macrophages pre-incubated with 10 μg/ml of the PU1 NP, PU2 NP, or PU3 NP exhibited decreased mRNA expression of IL-1β, IL-6, and TNF-α when compared with the cells treated only with LPS. This immunosuppression was even more prominent when 50 μg/ml of the PU NP was applied. This result indicates that the PU NP of the present invention possesses the anti-inflammatory effect by inhibiting gene expression of the pro-inflammatory cytokines. While the three types of PU NPs showed no difference in the immunosuppressive activities at a lower dose, PU3 NP exhibited greater activity than the other two at a higher dose.

### Example 3

### Anti-Inflammatory Effect of the Carboxyl-Functionalized Polyurethane Nanoparticles Via Reducing Activated Nuclear Factor Kappa B (NF-κB)

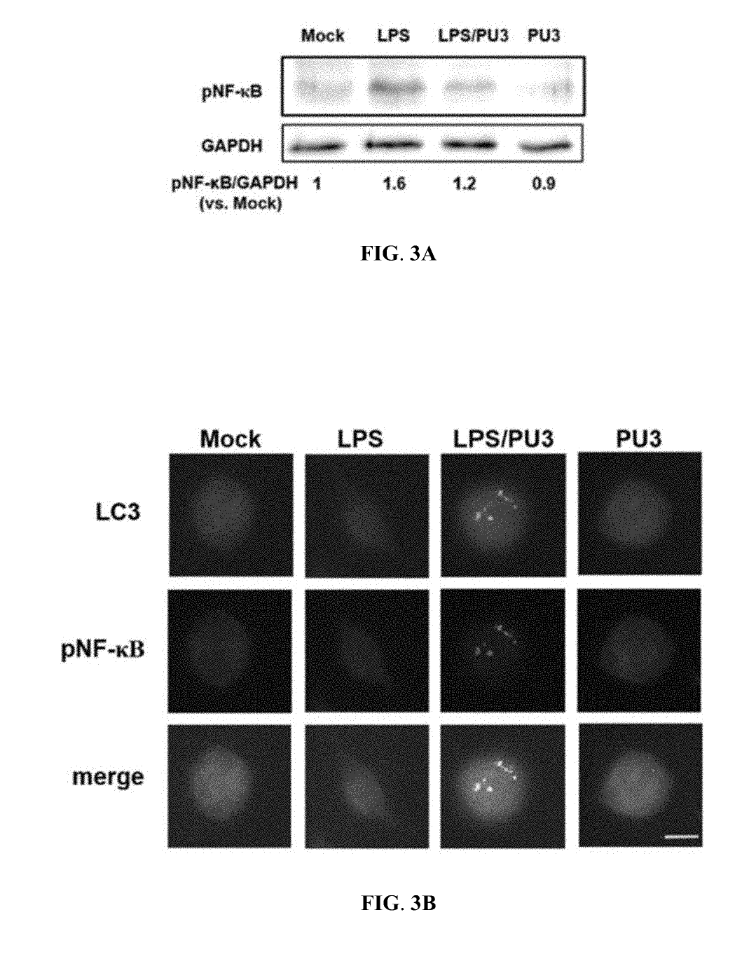

It has been reported that activated NF-κB, in the form of phospho-NF-κB (pNF-κB), regulates production of pro-inflammatory cytokines. To further verify the anti-inflammatory effect of the PU NP of the present invention, the levels of pNF-κB were analyzed by western blot after J774A.1 macrophages were incubated with PU3 NP (50 μg/ml), a preferred example of the PU NP of the present invention, for 30 minutes and treated with LPS (100 ng/ml) for 24 hours. As shown in FIG. 3A, LPS caused increased pNF-κB, but PU3 NP treatment inhibited this pNF-κB activation, indicating that the PU NP of the present invention effectively reduced the pNF-κB-related inflammation.

The down-regulation of the LPS-induced pNF-κB may be resulted from autophagy-lysosomal degradation of pNF-κB, which is stimulated by Ca²⁺ absorption by carboxyl groups of PU3 NP, transportation of Ca²⁺ into macrophages, and the resulting enhanced autophagy. To identify the possible role of autophagy in NF-κB regulation, the co-localization of pNF-κB and microtubule-associated protein 1 light chain 3 (abbreviated as LC3), a marker of autophagosome, was examined by fluorescence microscopy.

FIG. 3B shows fluorescence microscopy images of macrophages treated with LPS (100 ng/ml), PU3 NP (50 μg/ml), or a combination of LPS and PU3 NP. According to FIG. 3B, stronger immunofluorescence staining which demonstrated autophagosome formation and co-localization of pNF-κB and LC3 were observed only in the LPS/PU3 group, while a weak and diffuse cytoplasmic staining was observed in other groups. The results indicate the PU NP of the present invention possesses the anti-inflammatory effect by reducing activated NF-κB.

### Example 4

### Anti-Inflammatory and Neuroprotective Effects of the Carboxyl-Functionalized Polyurethane Nanoparticles in a Rat Model of Acute Subdural Hemorrhage (SDH)

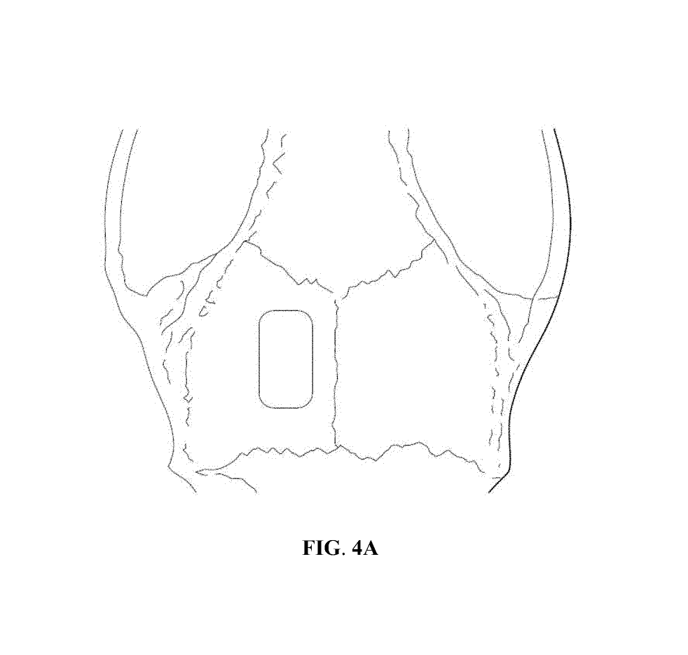

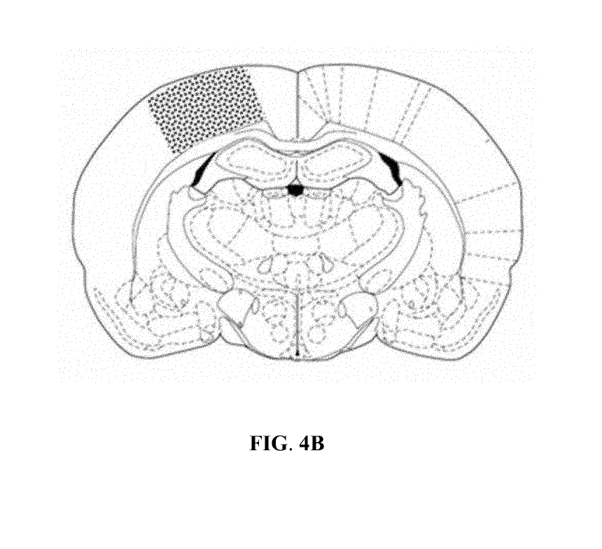

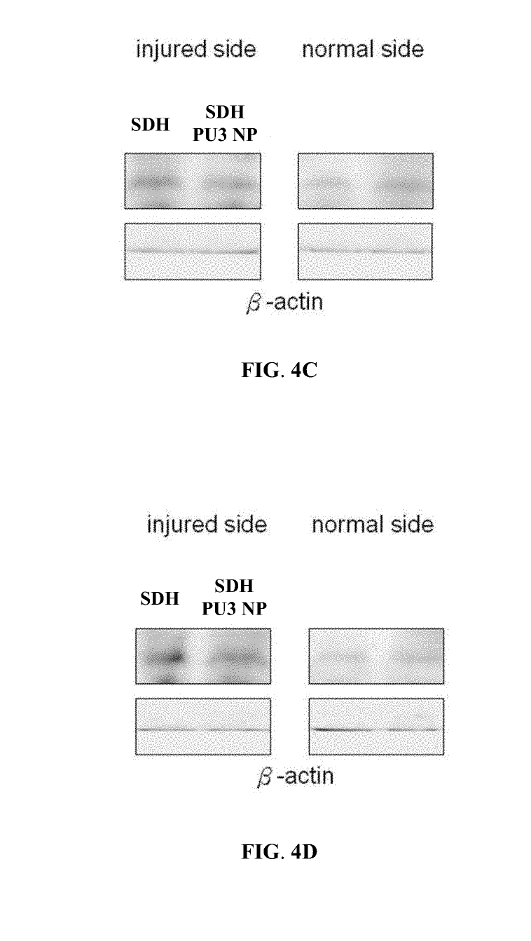

The clinical potential of PU NP of the present invention in suppressing ICH-induced inflammatory responses and promoting neural repair was assessed by using a rat model of SDH, the most common type of intracranial mass lesion in patients with severe head injuries. For experimentation, the rats were first placed into a stereotactic frame. After exposure of the skull, a 3×5 mm craniotomy was performed 1 mm left of and 1 mm posterior to the Bregma to form an rectangular opening, as shown in FIG. 4A. Next, a 23-gauge L-shaped blunted needle was inserted under the dura mater, and 400 μL of autologous venous blood from femoral vein was infused to cause SDH at rate of 50 μL/min. One hour post SDH, the clotted blood was evacuated as completely as possible. After clot removal, a solution of the PU3 NP (PU3 NP suspended in PBS at 360 mg/mL), a preferred example of the PU NP of the present invention, was directly dropped onto the exposed cortex with a SDH-induced injured area. As shown in FIG. 4B, the SDH-induced injured area is depicted as a dotted region in coronal section of a rat brain.

To verify the anti-inflammatory effect of the PU NP, western blot analysis was carried out to determine the levels of pro-inflammatory cytokines in the brains of the PU3 NP-treated rats killed 2 days post the SDH surgery, an example of minimally invasive neurosurgery. FIGS. 4C and 4D show expressions of TNF-α and IL-1β respectively in the injured or normal side of the rat cerebral cortex. Beta-actin (β-actin) was shown as the loading control. According to FIGS. 4C-4D, the cortex damaged by SDH exhibited higher TNF-α and IL-1β expressions in comparison with the normal cortex, suggesting SDH-induced inflammation. However, the PU3 NP treatment mildly reduced TNF-α and IL-1β expressions in the injured cortex. This result indicates the anti-inflammatory effect of the PU NP of the present invention.

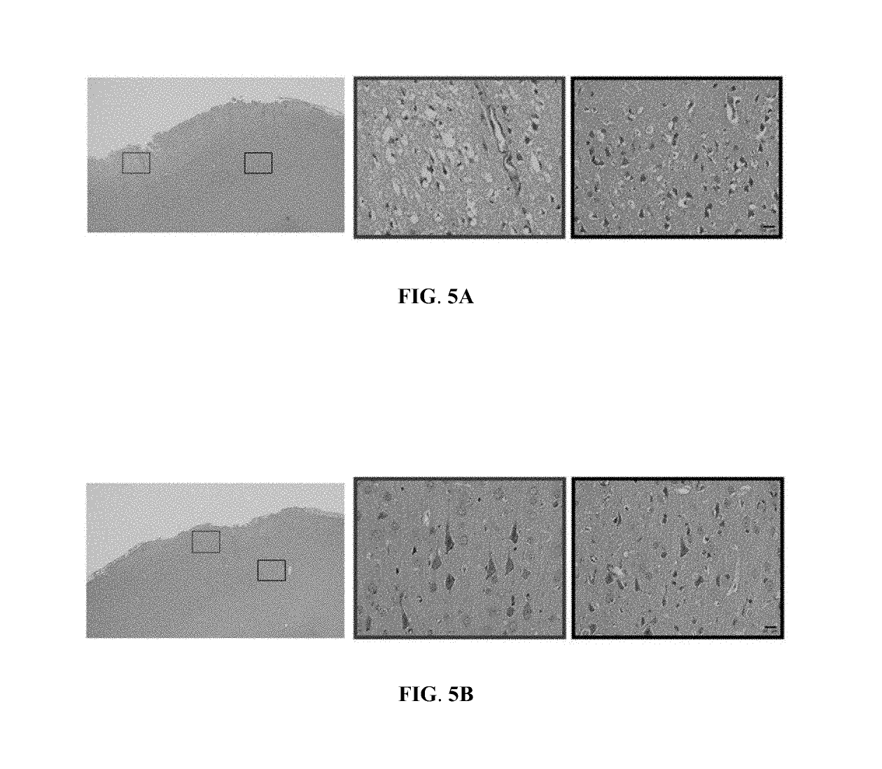

To verify the neuroprotective effect of the PU NP, histopathological examination of the rat cortex was conducted with H&E staining. As shown in FIG. 5A, the SDH surgery caused significant vacuolation and neuronal loss in the rat cortex. However, as shown in FIG. 5B, the PU3 NP treatment following SDH obviously recovered the SDH-related brain injuries. This result indicates the PU NP of the present invention promotes repair of the injured neural tissues.

### Example 5

### Neuroprotective Effect of the Carboxyl-Functionalized Polyurethane Nanoparticles in a Middle Cerebral Artery Occlusion (MCAO) Rat Model

The ameliorating effect of the PU NP of the present invention on neurological deficits was also evaluated in rats with ischemic stroke, occurring with blocked arteries to the brain and resulting in neuronal cell death. In this example, a MCAO rat model was established to mimic ischemic stroke. For experimentation, the right MCA of rats was reversibly ligated under a stereomicroscope. Both common carotid arteries were then occluded using non-traumatic aneurysm clips. After 60 minutes of ischemia, all arterial occlusions were released, resulting in reperfusion. In this model, ischemia for 60 minutes produces a large infarct confined to the right MCA cortex region with an ˜90% reduction in regional blood flow. After 1.5 or 3 hours of reperfusion, a solution of the PU3 NP (PU3 NP suspended in PBS at 360 mg/mL), a preferred example of the PU NP of the present invention, was intra-arterially injected via internal carotid artery. After 24 hours, the rats were sacrificed and brain slices were obtained and stained with TTC to measure the effect of the PU3 NP on the lesion size of the injured brain.

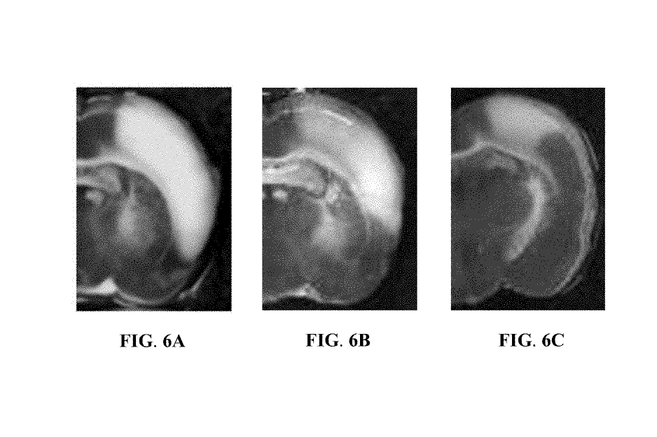

FIGS. 6A-6C show brain slices of the MCAO rat models without PU NP treatment (FIG. 6A), with PU3 NP treatment at a 20 mg/kg after 1.5 hours of reperfusion (FIG. 6B), or with PU3 NP treatment at a dose of 10 mg/kg after 3 hours of reperfusion (FIG. 6C). The white areas in the brain slices indicated the injured areas, such as the injured cortex. Compared to the rat model without PU NP treatment, the ones treated with PU3 NP exhibited reduced injured areas. This result indicates that the PU NP of the present invention facilitates repair of the injured neural tissues in ischemic stroke.

In conclusion, the method of the present invention effectively suppresses inflammation via inhibiting gene expression of pro-inflammatory cytokines, including IL-1β, IL-6, and TNF-α, and reducing activated NF-κB, and promotes repair of the injured neural tissue in a subject in need by administering to the injured neural tissue an effective amount of a carboxyl-functionalized polyurethane nanoparticle. The examples of the present invention demonstrate that the polyurethane nanoparticle with surface modification of the negatively charged carboxyl groups exerts the anti-inflammatory effect without carrying any other anti-inflammatory agents. Therefore, the method of the present invention may be directly applied during surgery such as a minimally invasive neurosurgery to reduce neuroinflammation, decrease neural injuries caused by brain surgery, lower the risk of infection due to systemic application of conventional anti-inflammatory drugs such as steroids, and facilitate neurological recovery of patients.

What is claimed is:

1. A method of promoting repair of an injured neural tissue in a subject in need thereof, comprising administering to the injured neural tissue an effective amount of a carboxyl-functionalized polyurethane nanoparticle, wherein the injured neural tissue is an injured cerebral cortex that is not injured from autophagy dysfunction.

2. The method of claim 1, wherein the carboxyl-functionalized polyurethane nanoparticle comprises a main chain of polyurethane comprising a hard segment and a soft segment, wherein the hard segment is formed by reaction of a diisocyanate and a chain extender, and the soft segment is a biodegradable oligodiol.

3. The method of claim 2, wherein the biodegradable oligodiol is polycaprolactone diol.

4. The method of claim 2, wherein the diisocyanate is isophorone diisocyanate.

5. The method of claim 2, wherein the chain extender is selected from the group consisting of 2,2-bis(hydroxymethyl)propionic acid and ethylenediamine.

6. The method of claim 1, wherein the carboxyl-functionalized polyurethane nanoparticle has a zeta potential of about −70 to −50 mV.

7. The method of claim 1, wherein the carboxyl-functionalized polyurethane nanoparticle is at a size of about 34-64 nm.

8. The method of claim 1, wherein the carboxyl-functionalized polyurethane nanoparticle is administered at a dose of at least 10 mg/kg.

9. The method of claim 1, wherein the carboxyl-functionalized polyurethane nanoparticle is administered topically.

10. The method of claim 1, wherein the carboxyl-functionalized polyurethane nanoparticle suppresses gene expression of a pro-inflammatory cytokine.

11. The method of claim 10, wherein the pro-inflammatory cytokine is selected from the group consisting of interleukin-1 beta (IL-1β), interleukin-6 (IL-6), and tumor necrosis factor alpha (TNF-α).

12. The method of claim 1, wherein the carboxyl-functionalized polyurethane nanoparticle reduces activated nuclear factor kappa B (NF-κB).

13. (canceled)
